(19) United States
(12) Patent Application Publication
(10) Pub. No.: US 2021/0386344 A1
(43) Pub. Date: Dec. 16, 2021

(54) AN IMPROVED PSYCHOMETRIC TESTING SYSTEM

(71) Applicant: Anthony E.D. MOBBS, North Sydney, New South Wales (AU)

(72) Inventor: Anthony E.D. MOBBS, North Sydney, New South Wales (AU)

(21) Appl. No.: 17/292,155

(22) PCT Filed: Nov. 8, 2019
(86) PCT No.: PCT/AU2019/051233

§ 371 Date: May 7, 2021

(30) Foreign Application Priority Data
Nov. 8, 2018 (AU) 2018904267
May 27, 2019 (AU) 2019901802
Aug. 16, 2019 (AU) 2019902975

(51) Int. Cl. A61B 5/16; A61B 5/00; G06F 40/242; G06F 40/253; G06F 40/247; G06F 40/205; G10L 15/18; G10L 15/22; G06F 40/58; G06F 40/279; G10L 25/63; G16H 10/20; G16H 50/20; G06F 16/35; G06F 16/31

(52) U.S. Cl. CPC A61B 5/167; G06F 16/316; A61B 5/165; G06F 40/242; G06F 40/253; G06F 40/247; G06F 40/205; G10L 15/1822; G10L 15/22; G06F 40/58; G06F 40/279; G10L 25/63; G16H 10/20; G16H 50/20; G06F 16/353; A61B 5/4803

(57) ABSTRACT

The present invention provides a method of categorising words and/or text wherein the following steps are performed: a) compiling a catalogue of selected words of a language which are identified and selected from at least one dictionary and which are descriptive of intrapersonal behaviours and/or interpersonal interactions, and the selected words being of one of, or combinations of two or more of, or all of, the following types: verbs, adjectives, nouns and idioms (nouns may be descriptors of behaviour, personality or emotion); b) identifying synonyms for each one of the selected words from at least one thesaurus; c) identifying archetypal words from the respective groups of one selected word and its respective synonyms; d) rating the archetypal words with scores relating to affiliation and dominance thereby producing a matrix; e) applying ratings to all of the selected words and the synonyms.

### FIELD OF THE INVENTION

The present invention relates to: a method of categorising words and text; the use of a predefined 5 times 5 (5×5) matrix to assist in the categorisation of words and text; utilising such methods and matrices to provide a personality and or behaviour classification system; and a method of speech analytics, to categorise the personality and or behaviour of a speaker or writer; whether in English or other languages.

### BACKGROUND OF THE INVENTION

The research field of personality is rife with theories and taxonomies, but all the while there has been a need to identify a means of pinpointing human emotion, behaviour, and traits. Saucier and Goldberg (2003) identified that a major goal of personality research is to develop an overarching taxonomy capable of describing, delineating, and organising single-word descriptors of personality.

Any reference herein to known prior art does not, unless the contrary indication appears, constitute an admission that such prior art is commonly known by those skilled in the art to which the invention relates, at the priority date of this application.

### SUMMARY OF THE INVENTION

The present invention provides a method of categorising words and/or text wherein the following steps are performed:
a) compiling a catalogue of selected words of a language which are identified and selected from at least one dictionary and which are descriptive of intrapersonal behaviours and/or interpersonal interactions, and the selected words being of one of, or combinations of two or more of, or all of, the following types: verbs, adjectives, nouns and idioms (nouns may be descriptors of behaviour, personality or emotion);
b) identifying synonyms for each one of the selected words from at least one thesaurus;
c) identifying archetypal words from the respective groups of one selected word and its respective synonyms;
d) rating the archetypal words with scores relating to affiliation and dominance thereby producing a matrix;
e) applying ratings to all of the selected words and the synonyms.

The matrix can be one of: three by three or a five by five or seven by seven, or three by five, or three by seven, or five by seven.

The matrix can be such that when it includes an axis of three, has index values of −1, 0, +1; and or when it has and axis of five, has index values of −2, −1, 0, +1, +2; and or when it has an axis of seven, has index values of −3, −2, −1, 0, +1, +2, +3.

There can be a five by five matrix, and has indexes of −2, −1, 0, +1, +2.

The method can be modified by synonyms being replaced by antonyms.

The present invention provides a method of categorising words and/or text wherein the following steps are performed:
a) compiling a catalogue of selected words of a language which are identified and selected from at least one dictionary and which are descriptive of intrapersonal behaviours and/or interpersonal interactions, and the selected words being of one of, or combinations of two or more of, or all of, the following types: verbs, adjectives, nouns and idioms;
b) identifying antonyms for each one of the selected words from at least one thesaurus;
c) identifying archetypal words from the respective groups of one selected word and its respective antonyms;
d) rating the archetypal words with scores relating to affiliation and dominance thereby producing a matrix;
e) applying ratings to all of the selected words and the antonyms.

The matrix can be one of: three by three or a five by five or seven by seven, or three by five, or three by seven, or five by seven.

The matrix can be such that when it includes an axis of three, has index values of −1, 0, +1; and or when it has and axis of five, has index values of −2, −1, 0, +1, +2; and or when it has an axis of seven, has index values of −3, −2, −1, 0, +1, +2, +3.

There can be a five by five matrix, and has indexes of −2, −1, 0, +1, +2.

The antonyms can be in a 5×5 matrix.

The antonyms can be selected from said matrix by being separated by at least one index unit on at least one of the X-axis and/or Y-axis.

The antonyms can be used in a test regarding personality and or behaviour and or emotion.

A subject of the test can be provided by the antonyms and is asked for a reaction to them, through one or more than one of: an app, an application, a phone, a mobile device, a web based application, a website, a paper based questionnaire.

The present invention provides a five by five matrix for categorising words of a language, the matrix comprising orthogonal axes of affiliation and dominance, the axes being indexed −2, −1, 0, +1, +2. The matrix can be produced by the method of paragraphs [004] to [012].

The present invention also provides a personality and or behaviour classification system comprising analysis of the words utilised or parsed by a subject, the system including testing the subject to collect parsed words or collecting the words (by voice to text or transcripts) and or writings of the subject, analysing the utilised or parsed words by means of the categorising method of any one of claims 5 to 9, whereby the utilised or parsed words are the selected words and or the antonyms of the selected words.

The system can have the words provided by a subject through one or more than one of: an app, an application, a phone, a mobile device, a web based application, a website, or a paper based questionnaire.

The words can be collected by voice to text or transcripts.

The system can include reducing voice to text, or review of transcripts of the speech, and applying the method or matrix to key words used in the text and or transcript.

The speech or words can be in a language other than the language used in the method or matrix, and the words can be translated into the language used in the method or matrix.

The language, dictionary and or thesaurus is, or is applicable to, one of the following languages: English, French, German, Spanish, Portuguese, Chinese, Japanese, Korean, Indian, Arabic, Greek, or any other language translatable by Google Translate.

The present invention also provides a method of analysing speech by means of the method or matrix described above, the method including reducing voice to text, or review of transcripts of the speech, and applying the method or matrix to key words used in the text and or transcript.

When the speech is in a language other than the language used in the method or matrix, the text or the transcript is translated into the language used in the method or matrix.

The language, dictionary and or thesaurus is, or is applicable to, one of the following languages: English, French, German, Spanish, Portuguese, Chinese, Japanese, Korean, Indian, Arabic, Greek, or any other language translatable by Google Translate.

A two axis matrix for use in a psychometric test or personality and or behaviour classification system, said matrix comprising orthogonal axes where a central location is occupied by a neutral expression or word.

A two axis matrix as claimed in claim 25, wherein said matrix is one of: three by three or a five by five or seven by seven, or three by five, or three by seven, or five by seven.

A psychometric test or a personality and or behaviour classification system comprising analysis of words utilised or parsed by a subject, said system utilising a two axis matrix as claimed in any one of claim 25 or 26.

A system as claimed in claim 27, wherein test or system is provided to a subject through one or more than one of: an app, an application, a phone, a mobile device, a web based application, a website, or paper based form.

### BRIEF DESCRIPTION OF THE DRAWINGS

A detailed description of a preferred embodiment will follow, by way of example only, with reference to the accompanying figures of the drawings, in which:

FIG. 1 represents a kernel density plot of catalogued words;

FIG. 2 represents a single kernel density plot for the two words ‘honest’ and ‘dishonest’, put on a single plot for comparison purposes, with concentric shapes or iso-lines representing the location of synonyms and the white triangle represents the judges' score for the word ‘dishonest’, and the white circle represents the judges' score for ‘honest’;

FIG. 3 represents kernel density plots for the ten DSM-5 Personality Disorders and each of the dipoles that define the five factors of the NEO-PI-R Five Factor Model (Costa & McCrae, 1992);

FIG. 4 represents kernel density plots for the twenty antonymic dipoles referred to in Part 4 below;

FIG. 5 represents kernel density plots for good and bad leadership behaviours and criminal behaviours identified by Allison et al (2012); Bass (2008); and Kellerman (2004);

FIG. 6A represents kernel density plots for speeches of Churchill at a variety of times in his career;

FIG. 6B represents kernel density plots for speeches of Hitler pre-start of war and post-start of war;

FIG. 6C (located below FIG. 3 in the drawing sheets) is a Venn diagram of indicated behavioural therapies for individuals with personalities in each region of the topology;

FIG. 7 is a 5×5 matrix with a Northwest to Southeast diagonal double ended arrow including the (0, 0) cell with arrow heads indicating from where antonymic words are picked;

FIG. 8 is a 5×5 matrix with a North-Northwest to South-Southeast diagonal double ended arrow including the (0, 0) cell with arrow heads indicating from where antonymic words are picked;

FIG. 9 is a 5×5 matrix with a North to South diagonal double ended arrow through the centre 5 cells including the (0, 0) cell with arrow heads indicating from where antonymic words are picked;

FIG. 10 is a 5×5 matrix with a North-Northeast to South-Southwest diagonal double ended arrow including the (0, 0) cell with arrow heads indicating from where antonymic words are picked;

FIG. 11 is a 5×5 matrix with a Northeast to Southwest diagonal double ended arrow through 5 cells including the (0, 0) cell with arrow heads indicating from where antonymic words are picked;

FIG. 12 is a 5×5 matrix with a West-Northwest to East-Southeast diagonal double ended arrow including the (0, 0) cell with arrow heads indicating from where antonymic words are picked;

FIG. 13 is a 5×5 matrix with a Northwest to Southeast diagonal double ended arrow through 3 cells including the (0, 0) cell with arrow heads indicating from where antonymic words are picked;

FIG. 14 is a 5×5 matrix with a North to South double ended arrow through 3 cells including the (0, 0) cell with arrow heads indicating from where antonymic words are picked;

FIG. 15 is a 5×5 matrix with a Southwest to Northeast diagonal double ended arrow through 3 cells including the (0, 0) cell with arrow heads indicating from where antonymic words are picked;

FIG. 16 is a 5×5 matrix with a West-Southwest to East-Northeast diagonal double ended arrow including the (0, 0) cell with arrow heads indicating from where antonymic words are picked;

FIG. 17 is a 5×5 matrix with a West to East double ended arrow through 5 centre cells including the (0, 0) cell with arrow heads indicating from where antonymic words are picked;

FIG. 18 is a 5×5 matrix with a West to East double ended arrow through 3 centre cells including the (0, 0) cell with arrow heads indicating from where antonymic words are picked;

FIG. 19 is a 5×5 matrix with a North to South double ended arrow through 5 first (from left side) column cells with arrow heads indicating from where antonymic words are picked;

FIG. 20 is a 5×5 matrix with a North to South double ended arrow through 5 second (from left side) column cells with arrow heads indicating from where antonymic words are picked;

FIG. 21 is a 5×5 matrix with a North to South double ended arrow through 5 third or middle (from left side) column cells with arrow heads indicating from where antonymic words are picked;

FIG. 22 is a 5×5 matrix with a North to South double ended arrow through 3 first (from left side) column cells with arrow heads indicating where antonymic words are picked from;

FIG. 23 is a 5×5 matrix with a North to South double ended arrow through 3 second (from left side) column cells with arrow heads indicating from where antonymic words are picked;

FIG. 24 is a 5×5 matrix with a North to South double ended arrow through 3 third or middle (from left side) column cells with arrow heads indicating from where antonymic words are picked;

FIG. 25 is a 5×5 matrix with a North to South double ended arrow through 5 fourth (from left side) column cells with arrow heads indicating from where antonymic words are picked;

FIG. 26 is a 5×5 matrix with a North to South double ended arrow through 5 fifth (from left side) column cells with arrow heads indicating from where antonymic words are picked;

FIG. 27 is a 5×5 matrix with a North to South double ended arrow through 3 fourth (from left side) column cells with arrow heads indicating from where antonymic words are picked;

FIG. 28 is a 5×5 matrix with a North to South double ended arrow through 3 fifth (from left side) column cells with arrow heads indicating from where antonymic words are picked;

FIG. 29 is a 5×5 matrix with a West to East double ended arrow through 5 first (from top) row of cells with arrow heads indicating from where antonymic words are picked;

FIG. 30 is a 5×5 matrix with a West to East double ended arrow through 5 second (from top) row of cells with arrow heads indicating from where antonymic words are picked;

FIG. 31 is a 5×5 matrix with a West to East double ended arrow through 5 third or middle (from top) row of cells with arrow heads indicating from where antonymic words are picked;

FIG. 32 is a 5×5 matrix with a West to East double ended arrow through 3 first (from top) row of cells with arrow heads indicating from where antonymic words are picked;

FIG. 33 is a 5×5 matrix with a West to East double ended arrow through 3 second (from top) row of cells with arrow heads indicating from where antonymic words are picked;

FIG. 34 is a 5×5 matrix with a West to East double ended arrow through 3 third or middle (from top) row of cells with arrow heads indicating from where antonymic words are picked;

FIG. 35 is a 5×5 matrix with a West to East double ended arrow through 5 fourth (from top) row of cells with arrow heads indicating from where antonymic words are picked;

FIG. 36 is a 5×5 matrix with a West to East double ended arrow through 5 fifth (from top) row of cells with arrow heads indicating from where antonymic words are picked;

FIG. 37 is a 5×5 matrix with a West to East double ended arrow through 3 fourth (from top) row of cells starting at the first column on the left with arrow heads indicating from where antonymic words are picked;

FIG. 38 is a 5×5 matrix with a West to East double ended arrow through cells in fifth (from top) row starting at the first column on the left with arrow heads indicating from where antonymic words are picked;

FIG. 39 is a 5×5 matrix with a diagonal or angled orientation double ended arrow from third (from top) row of cells and second column (from left) to the first or top row and third column (from the left) with arrow heads indicating from where antonymic words are picked;

FIG. 40 is a 5×5 matrix with a diagonal or angled orientation double ended arrow from first or top row of cells and first column to the second (from top) row fourth column from left with arrow heads indicating from where antonymic words are picked;

FIG. 41 is a density plot of a test result from a test as described in PART 8, as answered by an unknown person;

FIG. 42 is a representation of a web page with the antonymic word separated by NEITHER as described in Part 8 below; and

FIG. 43 shows the binary pairs maximising overall contrast of the psychological test of Part 12.

### DETAILED DESCRIPTION OF THE EMBODIMENT OR EMBODIMENTS

An embodiment will now be described which can generally be described as an overarching taxonomy expressed in two linguistically-based dimensions which would allow for existing constructs to be compared and contrasted visually, in the same way that an atlas aids geographic visualisation. The present invention proposes, a two-dimensional lexical model that is capable of mapping a created catalogue of verbs, adjectives, nouns and idioms that are descriptive of personality and interpersonal behaviour. When applied to a range of existing psychological, psychiatric, sociological, educational, cultural and ethical constructs, distinct visual delineations between the various concepts are observed, resembling an atlas.

The taxonomy that describes the entirety of a subject may be described as a topology, provided that two essential criteria are met. The first criterion is that the dimensions (axes) are orthogonal (perpendicular), for example, the geographical atlas' lines of longitude (East-West) and latitude (North-South). Researchers have criticized previous taxonomies of personality for selecting or deriving non-orthogonal dimensions. The present invention avoids such criticisms, and satisfies the first criterion of topologies, by proposing the linguistic orthogonal dimensions of ‘affiliation’ and ‘dominance’, which are orthogonal.

The proposed dimensions of affiliation and dominance each have precedence within previous systems. Researchers have noted that the love-hate and power-weakness dichotomies describing personality and emotion have existed cross-culturally and in various forms since antiquity. Subsequently, the synonymic concepts of affiliation and dominance were selected as the dimensions of a circumplex model. A range of other circumplex models of personality and emotion with the same or synonymic dimensions have been suggested. Communion and agency are similar to affiliation and dominance but were defined as correlated concepts and are therefore non-orthogonal. Dominance and affiliation have also been recognised as primary dimensions of behaviour in non-human primates, hyenas, birds and fish which suggests a role for topological mapping across vertebrate taxa.

The second criterion for a taxonomy to be considered a topology is that the axes must be divisible into non-overlapping categories. The proposed inventive system segments the dimensions of affiliation and dominance into an odd number of non-overlapping categories, thus creating a square matrix or grid. Whilst the constructs of affiliation and dominance have previously been referenced by circumplex models, they were defined radially. As a result, previous circumplex and two-dimensional models of personality, emotion and behaviour have failed to account for intrapersonal or neutral behaviours which score zero for either affiliation or dominance. An odd number of categories in the proposed inventive system allows for intrapersonal (affiliation score=0) as well as interpersonal behaviours (affiliation score #0).

Dimensions that are divided into distinct categories are known as ‘discrete’ whereas dimensions that may be infinitely divisible are known as ‘continuous’. Although many scales are continuous, they may be discreetly approximated for the purpose of utility. For example, temperature is a continuous scale but commonly expressed as an integer rounded to the nearest degree for convenience. Similarly, the proposed inventive system expresses the continuous dimensions in a discrete manner to the nearest whole number.

TABLE 1
Categorisation of actor behaviour into non-overlapping
categories of affiliation and dominance:

| | Affiliation | Dominance |
|---|---|---|
| 3 | The actor asserts, evokes, proffers or induces | The actor asserts, evokes, proffers or induces |
| | permanent affiliation with others | permanent dominance over others |
| 2 | The actor asserts, evokes, proffers or induces | The actor asserts, evokes, proffers or induces |
| | enduring affiliation with others | enduring dominance over others |
| 1 | The actor asserts, evokes, proffers or induces | The actor asserts, evokes, proffers or induces |
| | transient affiliation with others | transient dominance over others |
| 0 | The actor's behaviour is neutral | The actor's behaviour is neutral |
| −1 | The actor asserts, evokes, proffers or induces | The actor asserts, evokes, proffers or induces |
| | transient disaffiliation with others | transient submission to others |
| −2 | The actor asserts, evokes, proffers or induces | The actor asserts, evokes, proffers or induces |
| | enduring disaffiliation with others | enduring submission to others |
| −3 | The actor asserts, evokes, proffers or induces | The actor asserts, evokes, proffers or induces |
| | permanent disaffiliation with others | permanent submission to others |

While seven division category scales are indicated above, it is preferred that there be a selection of the five division category scale (−2, 4, 0, 1, 2), as it was deemed better than three (−1, 0, 1) and seven (−3, −2, −1, 0, 1, 2, 3) division category scales. Combining transient and enduring behaviours achieves a three division category scale version of the topology which may be appropriate for educational purposes. Differentiating reversible and irreversible behaviours, such as killing, achieves a seven category scale version of the topology, however in practice the outer extremities were sparse and of little utility, except perhaps in clinical applications. The five category scale was preferred due to the ease of definition, the absence of sparsity and the clear separation of the transient and enduring outcomes.

If it is desired to reduce to three non-overlapping categories for the affiliation dimension or the dominance dimension, then the preferred groupings from 7 dimensions are that categories +1, +2 and +3 are grouped together to form +1, and −1, −2 and −3 are grouped together to form −1, whereas from 5 dimension, the categories +1 and +2 are grouped together to form +1, and −1 and −2 are grouped together to form −1.

The concept of personality is defined by individual variations and interplay of thought, emotion and behaviour. Personality research has been previously criticised for restricting analysis to adjectives, neglecting the analysis of verbs and nouns which could bear information about behaviour more broadly. The present invention addresses this by cataloguing all dictionary-defined verbs, adjectives, nouns, idioms and emotions that relate to intrapersonal and interpersonal behaviour. To further broaden the topology, the catalogue includes intra- and interpersonal behaviours specific to individuals, dyads and groups such as families, corporations and nation states. The catalogue consists of almost 18,500 words in total. The categorisation of each word in the catalogue was semi-automated using a novel and replicable visualisation method that used the synonyms from two widely available thesauri.

The proposed orthogonal, two-factor topology and semi-quantitative methodology of the present invention forms a dynamic, sensitive and specific linguistic system able to classify individual words, word-pairs, and sentences descriptive of personality and behaviour. The methodology of the present invention can be used as a retrospective, prospective or real-time, dynamic socio-linguistic tool. It allows rapid, sensitive, and specific comparisons between existing taxonomies, is readily subject to scrutiny, and facilitates better understanding of broad cultural and social phenomena. The inherent diversity of language, responsive to evolutionary and cultural influences, has been semi-quantified and synthesised, carrying potential for both subject- and observer-led feedback and quantitative refinement. Therefore, Saucier and Goldberg's imperative of creating a unifying theoretical framework in personality research has been satisfied.

A range of existing psychological, sociological, educational and ethical constructs were categorised using the topology. These were visualised using kernel density plots which have a similar purpose to cartographic elevation maps. It was found that the translation of these existing taxonomies to the proposed topology was thorough, rapid and unambiguous. Where the constructs consisted of an antonymic dipole, for example love-hate, it was found that each pole was able to be visually distinguished using the topology, thus enabling application to a comprehensive range of existing constructs, and reaffirming the function of the topology as something which is akin to an atlas.

### PART 1: Preparation of Comprehensive Catalogue of Relevant Words

The objective of this Part was to prepare a comprehensive catalogue of dictionary-defined English-language verbs, adjective, nouns, emotions and idioms that are descriptive of personality and interpersonal behaviour. This catalogue will then be used as the basis for the rest of the paper and for the new topology.

Part 1: Method

The method is similar to the lexical analysis performed by others who scanned the entire contemporary dictionary, except that rather than paper dictionaries the present study used open source word repositories and proprietary online thesauri.

The procedure adopted was to:
STEP 1: Scan two open-source repositories of words to manually collate a preliminary list or catalogue or database. Wordnet (Princeton University, 2010) and Moby Part-of-Speech II (Ward, 2002a) with 155,287 and 233,356 words respectively were selected. When combined, there were many unique words. This process can be automated if required.
STEP 2: Scan the synonyms of all words identified in the first phase. Resources for this process were Oxford Thesaurus (Oxford University Press, 2017) and Merriam-Webster Thesaurus (Merriam-Webster Incorporated, 2018). The most preferred method used to perform this step was to use a python program to lookup the synonyms of all words in the catalogue in the Oxford and Merriam-Webster thesauri. The program tabulates the number of times each synonym occurs and then orders the list of synonyms in descending order of frequency. The synonyms that occur many times have a high likelihood of being suitable for inclusion in the catalogue. The synonyms were manually reviewed and either added to the catalogue or excluded. This process was repeated until no further words were identified for inclusion in the thesaurus. Of the 18,501 words in the catalogue, approximately 17,800 were identified using this method. An additional 700 words were added to the catalogue being technical terms used by clinicians to describe behaviours and personality types, for example, agoraphobia, disinhibition and perseverative.
STEP 3: Compare the catalogue with collections of adjectives previously compiled by Allport and Odbert (1936) and Norman (1967). This step was performed last so as to avoid inherent biases from previous studies.

Words not included within the selected thesauri were excluded from the catalogue on the basis that they were likely to be regionally specific colloquialisms, archaic terms or vocationally specific terms.
STEP 4: All selected words were reviewed and deemed to be descriptive of intrapersonal behaviours and interpersonal interactions as defined by a word that demands or implies the existence of two (or more) individuals. For verbs, examples were: “S/he verb her/him”, “I verb her/him” and “they verb her/him”. For adjectives, “s/he is an adjective person”. For nouns, “s/he is a noun and always verbs him/her/them” and “their behaviour can be described as noun”. For emotions, “I feel a sense of emotion, so I verb”. The relevance of each word to describe personality and interpersonal behaviour was qualitatively verified by five judges; three clinical psychologists, a neurologist, and a tertiary psychology student.

Part 1: Results

A catalogue was precipitated which consisted of 18,501 words consisting of 3,039 verbs, 4,230 adjectives, 5,003 nouns and 6,229 idioms. 3,051 of the nouns were descriptors of emotion. The database of words is too voluminous for presentation in this document.

A comprehensive compilation of English-language verbs, adjective, nouns and emotions that are descriptive of personality and interpersonal behaviour was achieved in the catalogue. The number of adjectives identified in the present catalogue exceeds the most recently prepared catalogue by Ashton, Lee, and Goldberg (2004), who found 1,710 adjectives.

Since no previous catalogues of verbs, nouns or idioms exists, no comparisons could be made. With regard to the procedures adopted and the absolute number of words identified, the compilation was considered to be unbiased and sufficient for the purposes of subsequent Parts within this disclosure.

PART 2: Confirm that Catalogue has Affiliation and Dominance as Orthogonal Concepts.

Part 2: Method

As observed by Saucier and Goldberg (2003), the task of accurately and consistently classifying individual words was considered to be an ‘overwhelmingly complex problem’. To overcome this complexity, an automated approach was developed.

It was considered axiomatic that within the matrix topology synonyms should be tightly clustered whereas antonyms should be disparate. The automation process allowed the words to freely ‘move’ within the topology and come to rest at an equilibrium point where the forces of attraction between synonyms combined with the forces of repel between antonyms was minimised. It was further considered axiomatic that the force of attraction between synonyms should be proportional to the distance between them. For example, synonyms located on opposite sides of the topology should experience a strong force attracting the words closer together whereas synonyms located in the same cell of the topology should experience no force of attraction. The converse should be true for antonyms. These axioms are identical to the axioms underlying Hooke's law that describes the operation of springs. Given the extensive information contained in the reference thesauri, it was considered likely that a relatively small sample of words selected by experts would be sufficient to ‘seed’ the process with the overwhelming majority of words being allowed to find their resting, or equilibrium, position using an automated procedure.

The process utilised is:
STEP 1: Initialisation—Extract the synonyms and antonyms for each word in the catalogue from the Oxford Thesaurus and Merriam-Webster Thesaurus. This is best done by a Python computer programme to extract the words—an example of such a Python programme is provided in the Appendix 1.
STEP 2: Selection of archetypal words:
   a. The inventor used the topology to perform an initial coding of all words in the catalogue using dictionary definitions and synonyms. A combination of surveys and a Delphi process with ten judges was used to refine the original coding.
   b. Surveys of 25 individuals were performed to refine the initial selection.
   c. A modified Delphi process with ten judges was used to score 350 words which was used to further refine the initial selection. The judges were selected to ensure that at least one individual had specific expertise and personal experience in each region of the topology. Three of the judges had doctorates and considerable experience in psychology, medicine and education, one was a clinical psychologist, two were practicing lawyers, an elite athlete, a business executive, an individual with 30 years customer service experience and a graduate psychology student.
   d. Based upon the refined selection, the inventor selected the 125 words with the most synonyms from each cell and part of speech combination (approximately 4,113 words, 22% of the catalogue). These words were then collaboratively discussed and agreed by three clinical psychologists and a neurologist over three successive workshops. These words deemed to be the archetypal words for each cell of the inventive matrix.
STEP 3: First Iteration
   a. A Python computer program was developed to implement the following steps.
   b. Uncoded words in the catalogue that had 100% (threshold) of their synonyms previously coded were detected and encoded using step c.
   c. For each uncoded word, the equilibrium position was calculated such that the forces of attraction and repulsion were minimised. The forces of attraction and repulsion were weighted in inverse proportion to the ratio of synonym and antonym relationships between words in the catalogue.
   d. Step b was repeated after reducing the 100% threshold by 1%. The threshold was then reduced by 1% incrementally until all words in the catalogue were encoded.
STEP 4: Subsequent Iterations:
   a. Noting that words coded in the first iteration were not able to take advantage of the relationships with words subsequently encoded, step 3.c. was repeated continuously until no further changes were identified.
STEP 5: Additional Archetypal Words
   a. A review of uncoded words revealed that the categorisation in Step 3 was prone to pulling words towards the top left hand corner due to the higher relative density of words in that region and the relative sparsity of words in other quadrants. To overcome this deficiency, the author selected additional candidate words for categorisation as archetypal which were accepted provided consensus was achieved by two independent judges. When the number of archetypal words reached 4,000, it was considered that the problem was overcome.

Part 2: Results

Examples of the archetypal words selected in step 1 are shown in Table 2. A total of 530,482 synonym and 94,798 antonym relationships were identified between the 18,501 words in the catalogue. Each word in the catalogue had an average of 28.6 synonymic relationships with other words in the catalogue.

TABLE 2
Examples of the archetypal words selected:

| | | Affiliation | | | | |
|---|---|---|---|---|---|---|
| Dominance | Part of Speech | −2 | −1 | 0 | 1 | 2 |
| 2 | Verb | kill | bully | master | organise | inspire |
| | Adjective | vicious | tough | superior | persuasive | fantastic |
| | Noun | savage | arrogance | great | leader | magnificence |
| | Emotions | rage | conceit | determination | dynamism | triumph |
| 1 | Verb | reject | fight | make | give | nurture |
| | Adjective | nasty | difficult | keen | agile | happy |
| | Noun | creep | argument | purpose | gentility | vitality |
| | Emotion | hate | annoyance | concern | enjoyment | generosity |
| 0 | Verb | isolate | delay | rest | welcome | collaborate |
| | Adjective | peculiar | cool | plain | kind | reliable |
| | Noun | recluse | blind | balance | kindred | intimate |
| | Emotion | confusion | worry | tranquility | warmth | affection |
| −1 | Verb | trembling | mope | snooze | conform | follow |
| | Adjective | sad | anxious | dull | humble | selfless |
| | Noun | naysayer | malaise | novice | compliance | devotee |
| | Emotion | anguish | sadness | boredom | meekness | belonging |
| −2 | Verb | suicide | collapse | slumber | capitulate | kowtow |
| | Adjective | morose | cowardly | passive | obedient | obsequious |
| | Noun | fatalist | incapacity | sufferance | subservience | sycophancy |
| | Emotion | melancholy | frailty | vulnerability | submissiveness | servility |

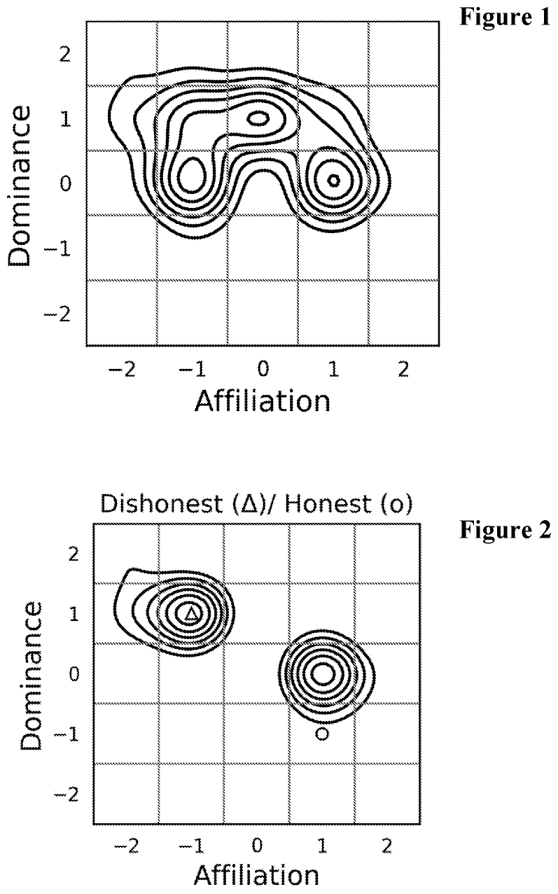

The frequency of words within each cell of the 5×5 matrix is shown in Table 3 and visualised in FIG. 1.

TABLE 3
Total encoded words according to cell within the 5 × 5
topological matrix (The weighted average affiliation and
dominance for the catalogue are −0.41 and 0.69 respectively):

| | Affiliation | | | | |
|---|---|---|---|---|---|
| Dominance | −2 | −1 | 0 | 1 | 2 |
| 2 | 716 | 580 | 685 | 232 | 368 |
| 1 | 1581 | 2966 | 3228 | 1682 | 163 |
| 0 | 748 | 2324 | 672 | 529 | 505 |
| −1 | 177 | 443 | 148 | 148 | 114 |
| −2 | 81 | 156 | 45 | 97 | 113 |

Example kernel density plots for the words ‘honest’ and ‘dishonest’ are seen using kernel density plots as illustrated in FIG. 2. The term ‘honest’ had 123 synonyms in the catalogue, of which 53(43%) had an affiliation score of 1 and a dominance of 0, with a further 63(51%) synonyms located in adjacent cells. The term ‘dishonest’ had 70 synonyms within the catalogue, of which 32(46%) had an affiliation score of −1 and a dominance of 1, with all remaining words in adjacent cells. The grey concentric circles are representative of the relative density of the aggregated Gaussian kernels and the white triangle and white circle represent the judges unblinded consensus coding. Visualisation was found to be most meaningful with the Gaussian kernels with a standard deviation of 0.45 and truncated at 1.28 standard deviations.

Given that the entire catalogue of words could be encoded using the topology, it is comprehensive and efficacious for lexical research. It is proposed that the orthogonal concepts of affiliation and dominance are universal and based in human evolution, and thus translation to other languages and broader application of the topology expected. The concentration of words in the top left quadrant (31%) compared to the top right hand corner (14%) was notable, and it is hypothesised that this weighting of language would have its basis in evolutionary survival as predicted by Darwin and others. Words describing non-dominant behaviours (dominance <0) accounted for 8% of the catalogue, and it was noted that many such words, for example ‘needy’, could either be used in an affiliative or disaffiliative manner depending upon the interpersonal context. Similarly, other words could be used in a dipolar dominant or non-dominant manner, for example, ‘cynic’ could be used in a dominant, ‘sardonic’, or non-dominant, ‘disenchanted’, context, albeit that it was always disaffiliative.

### PART 3: Application of the Topology

The objective of this Part was to apply the topology to leading psychological and psychiatric taxonomies to test whether the constructs within these taxonomies can be clearly visually delineated.

Part 3: Method

The Diagnostic and Statistical Manual of Mental Disorders (DSM-5) and Revised NEO Personality Inventory (NEO PI-R) were selected as illustrative, leading taxonomies of psychiatry and psychology respectively. The DSM-5 identifies ten unipole personality disorders, whereas the NEO PI-R identifies five constructs, each of which consist of a dipole with a positive and negative valence.

The constructs within each taxonomy were analysed to determine if they were defined in terms of single word descriptors or in sentences. For constructs defined in single words, the words descriptive of the construct were then collated. For example, ‘schizotypal personality disorder’ is defined in the DSM-5 using words such as ‘anxious’, ‘eccentric’ and ‘suspicious’. Each of these words were then coded according to the typology and visualised using plots to represent the construct of schizotypal personality disorder.

For constructs that were defined by sentences, the sentences were qualitatively reviewed by the three judges to determine the most appropriate single word which could clearly and thoroughly convey the meaning of the sentence. For example, one of the questions that measures agreeableness in the NEO-PI-R (Costa & McCrae, 1992) is “I believe that others have good intentions”. For this sentence, the word ‘trusting’ was determined to be the most appropriate single word descriptor listed in the catalogue.

Though this introduced subjectivity to the methodology, it was considered necessary for the purposes of confirming the comprehensiveness of the topology.

The words that comprise each of the constructs were plotted using kernel density plots in order to confirm whether the constructs may be visually delineated. Where the word descriptive of the DSM-5 or NEO PI-R construct was also found in the catalogue, for example ‘paranoid’, the construct was compared with the plot of the word in the catalogue and its synonyms.

Part 3: Results

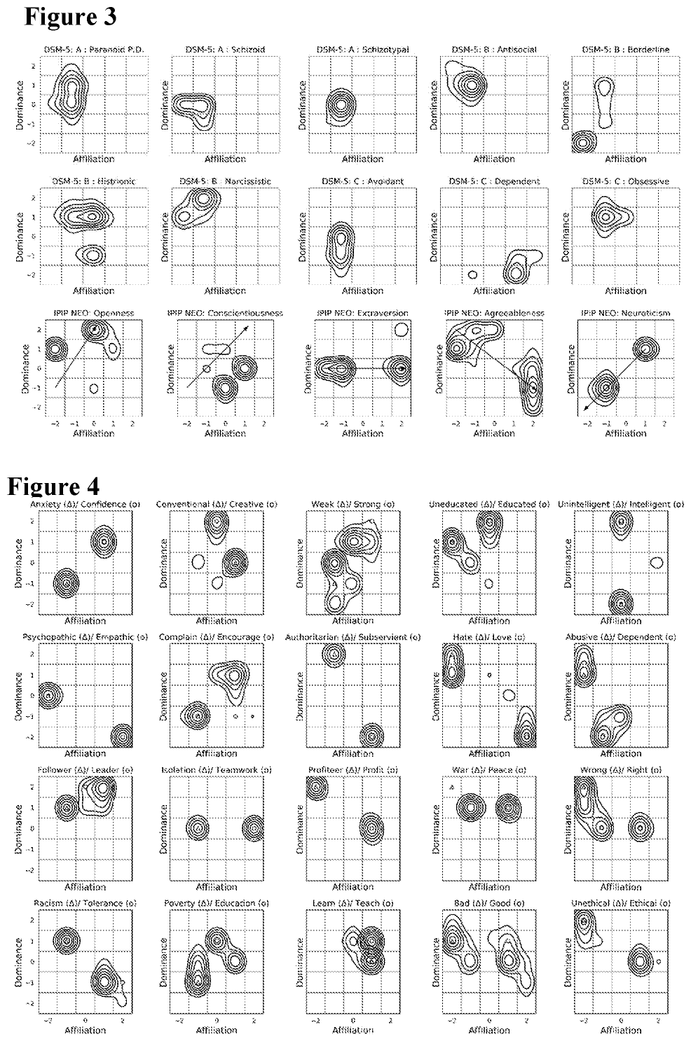

Each of the DSM-5 personality disorders was found to be visually delineated and discordant from each other, as were the dipoles for each of the five NEO-PI-R constructs (see FIG. 3). No significant differences between the DSM-5 and NEO PI-R constructs, and the equivalent dictionary definition, were identified with the exception of ‘openness’ which is defined by the NEO PI-R as ‘openness to ideas’. Honesty and frankness are synonyms of ‘openness’ in the Merriam-Webster Thesaurus. In all cases, the plots based on the topology were tightly clustered within a single cell. See FIG. 3.

The topology's distinct visual delineation of leading psychological and psychiatric constructs is a considerable advance beyond existing taxonomies of personality and behaviour. Furthermore, the topology enables comparison and discussion of these taxonomies with subsequent improved scope of research and clinical appraisal. Limitations of existing topologies can also be identified, for example, it is observed that the NEO-PI-R has an absence of traits shown in the top right hand corner of the grid, thus limiting its usefulness for assessing behaviours in this region.

The five constructs of the NEO-PI-R are often referred to as ‘dimensions’ of personality. Using the topology, these five ‘dimensions’ can be represented as five vectors on a two dimensional plane. Being vectors on a plane provides strong theoretical justification for the correlations empirically observed between these vectors (McCrae & Costa, 1987). The vectors representing agreeableness and neuroticism visually approximate orthogonality, and therefore should be uncorrelated. Conversely, the vectors representing openness and conscientiousness have a relatively acute angle between them indicating the likelihood of a strong correlation. Empirical studies are consistent with these theoretical predictions.

### PART 4: Testing Using Antonymic Dipoles

Part 2 demonstrated that encoding of the catalogue could be simplified by the use of synonyms enumerated within thesauri. However, antonyms were not included in this initial encoding process, and Part 4 aims to encompass these. Converse to the clustering or co-location of synonyms within topological categories, it is hypothesised that antonyms will be clearly visually delineated. The objective of this Part was to apply the method using antonymic dipoles.

Part 4: Method

For each word in the catalogue developed in Part 1, the antonyms were extracted from the Oxford Thesaurus and Merriam-Webster Thesaurus. The distance between each pole of the dipole was then calculated to identify instances where antonymic word pairs are co-located on the matrix. The common use antonymic word pairs, such as weak-strong, were selected in order to confirm the topology's scope of wider applicability, ranging from intrapersonal behaviours to the behaviours of nation states.

Part 4: Results

10,016 words in the catalogue did not have antonyms or did not have antonyms with other words in the catalogue. Of the 8485 words that did have antonym links to other words in the catalogue, 94,797 antonymic relationships were identified. Matrix co-locations due to identical scores were identified in only 360 words (1.6%). Examples of word pairs that were co-located were ask-answer, blunt-sharp and concerned-unconcerned. The average distance between antonymic word pairs was 2.3, median distance 2.2 and standard deviation 0.9. Twenty antonymic dipoles were plotted (see FIG. 4) demonstrating that the antonymic dipoles are visually distinct across a wide range of intrapersonal, interpersonal and societal interactions.

Approximately 98% of antonymic word pairs were delineated, that is not co-located, when visualised using the topology. For the 1.6% of antonymic pairs that were co-located, an analysis indicates that most instances the co-location was due to the words having multiple meanings depending upon context, for example the words ‘concerned’ and ‘unconcerned’ may be used affiliatively and disaffiliatively depending on the context. Other examples of words that are co-located are ‘begin’ and ‘finish’ and ‘blunt’ and ‘sharp’. Words with multiple or contextually specific meanings would generally be excluded from lexical research.

The clear visual delineation between antonymic words has verified the automated methodology in Part 2 to both consolidate synonymic words and delineate antonymic words. Single words and word pairs are easily interpretable within the topology and may be analysed in a variety of interpersonal settings for the purposes of personality research.

### PART 5: Personality Test Using Utilising the Topology as Well as the Antonymic Word Pairs

A significant purpose of taxonomies is the testing of personality to assist with clinical diagnosis and employment selection. The purpose of this Part is to detail a personality test utilising the topology with the antonymic word pairs identified in Part 4.

Part 5: Method

In Part 4, a total of 94,798 antonymic word pairs were identified. In order to select the most appropriate antonymic word pairs for assessment, the following criteria were used:
a. Preference was given to antonymic word pairs in which at least one word was located on the boundary of the matrix that is having an affiliation or dominance score of +2 or −2. This provided a more sensitive and specific personality test by spanning the matrix, compared to words that were more centralised. Pairs where one of the words was located at (0, 0) were excluded.
b. Preference was given to word pairs in which an opposing prefix was used to create the negative term. For example, ‘feeling’ and ‘unfeeling’ was preferred to ‘feeling’ and ‘insensitive’.
c. Preference was given to words with fewer syllables and single words rather than compound words, to maximise legibility and simplicity of interpretation.
d. A mixture of both verb-verb pair and adjective-adjective pairs were selected for use.

A survey of 50 antonymic word pairs formed the basis of an observer survey. The antonymic word pairs were selected so as to encompass the entire topology. In total, 20 subjects of either school or tertiary qualification aged 18-87 years, 10 male and 10 female, were invited to complete the survey. Each respondent was asked to complete the survey four times and instructed to think for 30 seconds prior to each test regarding the following people as directly known to them: the greatest leader, the unhappy person, the meanest person, a meek/kind person.

Part 5: Results

176 adjectival word pairs and 61 verbal word pairs were identified. The 20 antonymic word pairs selected were a-b, c-d, e-f etc.

NEO is a dipole test, however it is constructed from two unipoles. For example, for the test, by answering ‘no’ to one of the tests, it tells you nothing about whether the respondent will answer the questions for the negative pole.

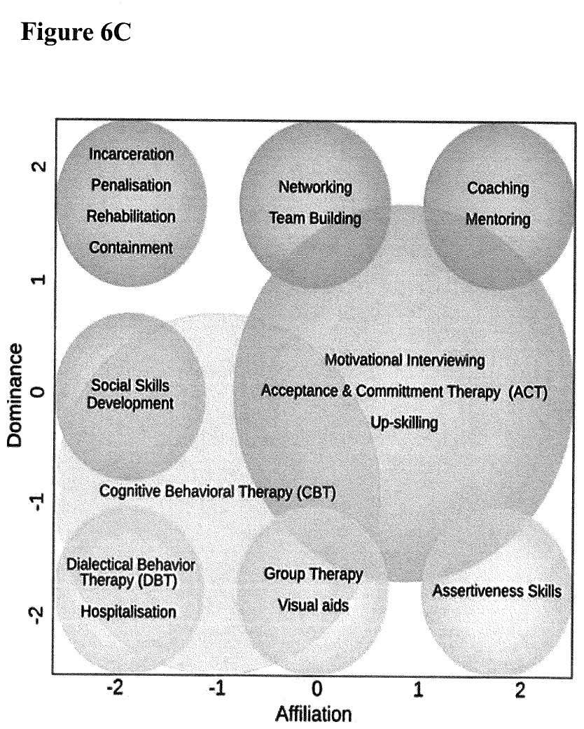

The new personality test has a number of advantages. It can be set up so that it takes only a few minutes to complete. Additionally, in the same way that the topology has been able to plot and differentiate existing psychological and psychiatric constructs, it can be that existing clinical treatments and societal responses to individuals presenting with identifiable personality types and habitual behaviours could be broadly mapped on the topology. FIG. 6C provides an overview in the form of a Venn diagram of indicated therapies for individuals whose personality and behaviours place them within each region of the topology. For disaffiliative/dominant behaviours (often criminal), the therapy (incarceration) is enforced.

Individuals motivated to self-assess their own behaviours and personality may be sufficiently motivated to self-inventory their current behaviours and identify future behaviours that they would like to develop.

The topology can similarly be used by individuals who wish to change their own behaviour or personality. By identifying their current behaviour, and identifying which cell of the matrix they wish to embody in the future, individuals can consider the behaviours of that cell a ‘checklist’ to work towards.

### PART 6: Antonymic Concepts of Good and Bad Leadership are Visually Delineated

The objective of this Part was to test whether the antonymic concepts of good and bad leadership can be visually delineated when plotted using the topology.

Part 6: Method Applied

Allison et al. (2012), Bass (2008) and Kellerman (2004) were selected as three leading textbooks of leadership. The three textbooks were read and behaviours associated with good and bad leadership were collated. The good and bad leadership behaviours were plotted as a dipole using the topology.

Single word descriptors of criminal behaviour were identified within the publicly available criminal and penal codes of Australia, New Zealand, Texas, California, England and Scotland as well as the Geneva Convention. The single word descriptors were plotted as a unipole using the topology.

Part 6: Results

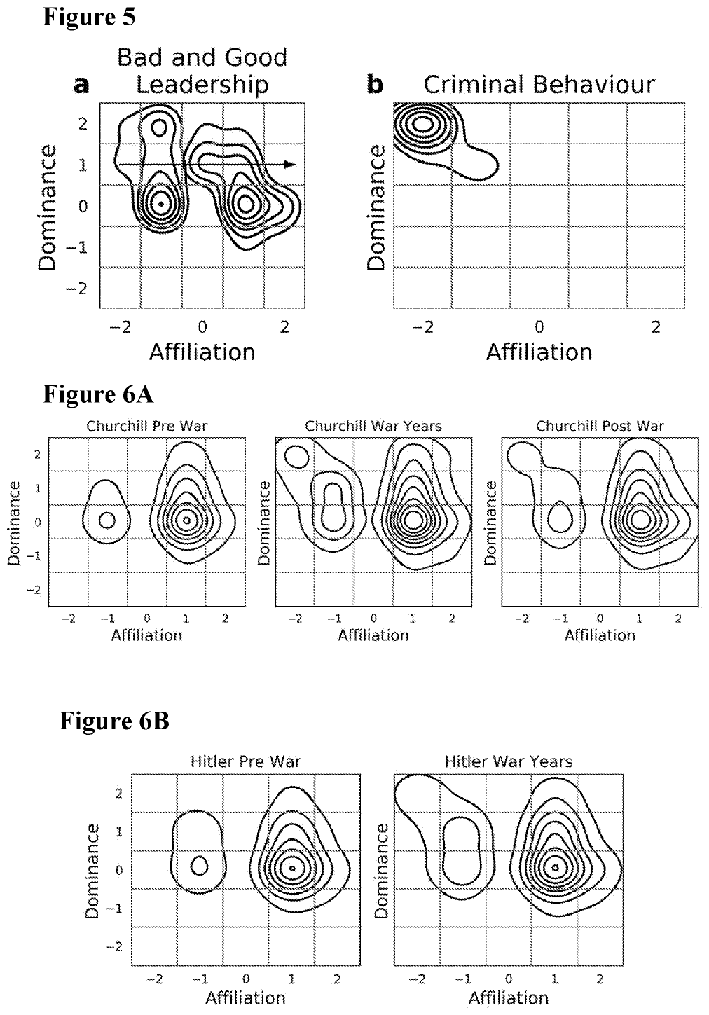

78 words descriptive of bad leadership behaviours were identified, for example, avoid, coerce and ignore. 61 words descriptive of good leadership behaviours were identified, for example, encourage, facilitate and mediate. The collections of words descriptive of good and bad leadership behaviours are shown in FIG. 5 demonstrating clear visual delineation between the concepts of good and bad leadership.

164 single word descriptors of criminal behaviour were identified, for example, murder, vandalize and rape. 34% of the collection of words were in cell (−2, 2) and a further 52% of words were in adjacent cells as visualised in FIG. 5.

Google scholar lists over 100,000 works published in 2017 referencing the subject of leadership, indicating the extensive interest in this fundamental organisational construct.

When viewed according to the topology, the difference between good and bad leadership behaviours is clearly distinguished. Bad leadership behaviours are, in summary, those that engender disaffiliation. Conversely, good leadership behaviours are those that engender affiliation or are associated with the intrapersonal processes of creativity, learning and development. Non-dominant behaviours are absent in both good and bad leadership, in other words, both good and bad leadership are either neutral or dominant.

Criminal behaviours are predominantly located in the upper left-hand corner of the matrix in which disaffiliative and dominant behaviours are co-located. No theoretical justification was able to be found that correlates criminal behaviours with dominant-disaffiliative behaviours. The use of the topology in identifying criminal behaviours and measuring the severity of such behaviours may be a future application of the topology.

The ability of the topology to visually distinguish the important real world constructs of good and bad leadership and the antonymic pair of leading and following (as demonstrated in Part 4) is a validation of the topology and indicates that it will be useful for a range of other real-world constructs. It is noteworthy that the concepts of good and bad leadership behaviours are closely aligned to the plots of ‘good’ and ‘bad’ plotted in Part 4. This suggests that good and bad topological profiles may have broader applicability to concepts such as culture, professional associations and family dynamics.

### PART 7: Automating the Meaning of Text

Automating the “meaning” of text has been a desirable goal of many organisations and systems.

Part 7: Method Applied

The speeches of two contemporary war-time leaders, Adolf Hitler and Winston Churchill, were selected for analysis. Using Python, the works of each author were broken into single words. Verbs of any tense were converted to the present tense for analysis.

Each word was coded according to the topology described in previous Parts above or excluded if not in the topology. Words with an affiliation score of zero were excluded. The ratio of affiliative and disaffiliative words were calculated.

Part 7: Results

The results of the application of this Part 7 method are illustrated in FIGS. 6A and 6B.

The ratio of affiliative to disaffiliative words for Hitler and for Churchill was derived where, after running and applying the methods above to the speeches of Churchill and Hitler, the following results were obtained:

| | | AFFILIATION | DOMINANCE |
|---|---|---|---|
| WHO | WHEN | RATIO | RATIO |
| WINSTON | PRE-WAR YEARS | 0.6 | 0.897 |
| CHURCHILL | | | |
| WINSTON | WAR YEARS | 0.477 | 0.914 |
| CHURCHILL | | | |
| WINSTON | POST WAR YEARS | 0.574 | 0.904 |
| CHURCHILL | | | |
| ADOLF HITLER | PRE-WAR YEARs | 0.503 | 0.912 |
| ADOLF HITLER | WAR YEARS | 0.456 | 0.912 |

It is noteworthy to observe from FIGS. 6A and 6B that both leaders use language that is disproportionately affiliative compared to the overall catalogue. The speech patterns for Hitler are reasonably stable over time whereas the patterns for Churchill show remarkable changes over the three periods examined. The changing speech patterns for Churchill are distinctly different. Hitler both pre-war and during the war years used two forms of leadership and language that broadly fits with the DSM-5 antisocial personality disorder.

The above described two-factor topology and semi-quantitative methodology has created a novel linguistic system able to classify individual words, word-pairs, and sentences descriptive of personality and behaviour. The above described topology allows rapid, sensitive and specific visual comparisons between existing taxonomies, is easily subject to scientific scrutiny, and facilitates better understanding of broad cultural and social phenomena.

The high density of words in the top left quadrant, representing disaffiliative and dominant behaviours may imply an interpersonal, if not evolutionary, importance of being able to identify and communicate such behaviours. Conversely, the relative paucity of words descriptive of non-dominant behaviours may suggest that there is a reduced evolutionary advantage in communicating these behaviours, though an individual may internally perceive them. Similarly, it is hypothesised that coordination within social groups may be facilitated by action towards individuals who display disaffiliative and subversive behaviours, either of redirection or exclusion. The topology may be used to facilitate future research spanning intrapersonal, intra- and inter-group settings. A neurobiological signature has been demonstrated in some personality traits, such as Machiavellianism (Cohen-Zimmerman et al. 2015) and it is proposed that the topology may be useful in identifying linguistic-behavioural neural correlates.

In addition to scientific application, the ability of the topology to clearly distinguish good and bad leadership behaviours and traits suggests a range of practical applications, in particular, leadership development might focus on both occupationally relevant behaviours and traits assessment. Equally, it is hypothesised that individuals ‘skilled’ in dominant, disaffiliative behaviour, such as bullying, may be diverted from anti-social behaviours by the learning and development of more socially constructive skills, disparate within the topology. The topology may be amenable to use in linguistic- and behaviour-based psychological or psychiatric interventions, such as cognitive and behavioural therapy.

By using contemporary verbs, adjectives, nouns and emotions to describe the full range of interpersonal behaviours, the topology tends to be socially dynamic and culturally responsive. The approach can be replicated in any language that has a thesaurus and replicated over time as thesauri are modified to reflect changing cultural norms.

### PART 8: Another Improved Psychometric Test:

Existing tests are generally in two forms: a) unipole and b) dipole. The test of PART 5 above is a dipole test. The following psychometric test is also a dipole test and is a variant of the test of Part 5.

Unipole testing consists of a number of questions that identify whether an individual has certain attributes or not. Typically, these tests count the number of questions answered in a manner that indicates existence of the condition. If the respondent's answers exceeds a predetermined threshold, then the respondent is said to have the condition. If the threshold is not met, then the respondent is considered not to have the condition.

Dipole tests generally consist of two unipole tests. Each of the two dipole are antonymically related, that is, one is the opposite of the other, for example, extroverted or introverted, neurotic or non-neurotic. Each of the two unipoles is generally independently measured using an equal number of questions. The unipole with the greater number of response indicating that condition is said to be the most prevalent state of the test respondent. Examples of this type of test are NEO PI-R (a Big-5 variant), Myers Briggs Type Indicator (MBTI).

Although each of the five dimensions of the NEO PI-R consists of a positive and negative valence dipole, the questions relating to each dipole pair are not in all cases strictly opposites, for example, the questions ‘I am always prepared’ and ‘I waste my time’ are presented within the NEO as the positive and negative valence questions for conscientiousness.

The use of antonymic dipoles combines the two questions required by the NEO into a single question by providing a scale. Furthermore, the use of single-word antonyms reduces the number of words considerably, further enhancing readability and clarity as to the response being sought by the researcher. In summary, the use of single-word antonymic dipoles reduces the number of questions required by half without any loss of associated specificity, sensitivity or power.

Both unipole and dipole tests are typically administered using Likert-type scales. Whilst Likert-type scales are popular, they have been criticised due to a range of biases that can be introduced. For example, central tendency bias, defensiveness bias and social desirability bias. Current tests typically use adjectival phrases to describe the concept being assessed, for example, “Worked hard when I was in school”, “Want to be the very best” and “Speak only when spoken to”.

The present invention envisages 12, 24, 36, 48, 60, 72, 84 and 96 question tests which use an alternative format to existing dipole tests. Each question within the test is presented as a dipole of two antonyms, for example, introvert-extrovert, bully-meek, open-closed etc. The antonyms may be verbs, adjectives or nouns. The proposed tests do not use adjectival phrases. The proposed test offers a third option being “NEITHER” to allow the respondent to only use a word that they consider to accurately match their behaviour and personality.

The antonym pairs selected will cover the entirety of the 25 cell 5×5 matrix so that many or most behaviours and personality descriptors are captured. The choice of antonyms is preferably decided upon as being that choice which maximises the ability to statistically discriminate between personality concepts. The antonyms pairs should also be selected so that each word is generally well recognised by the vast majority of the population.

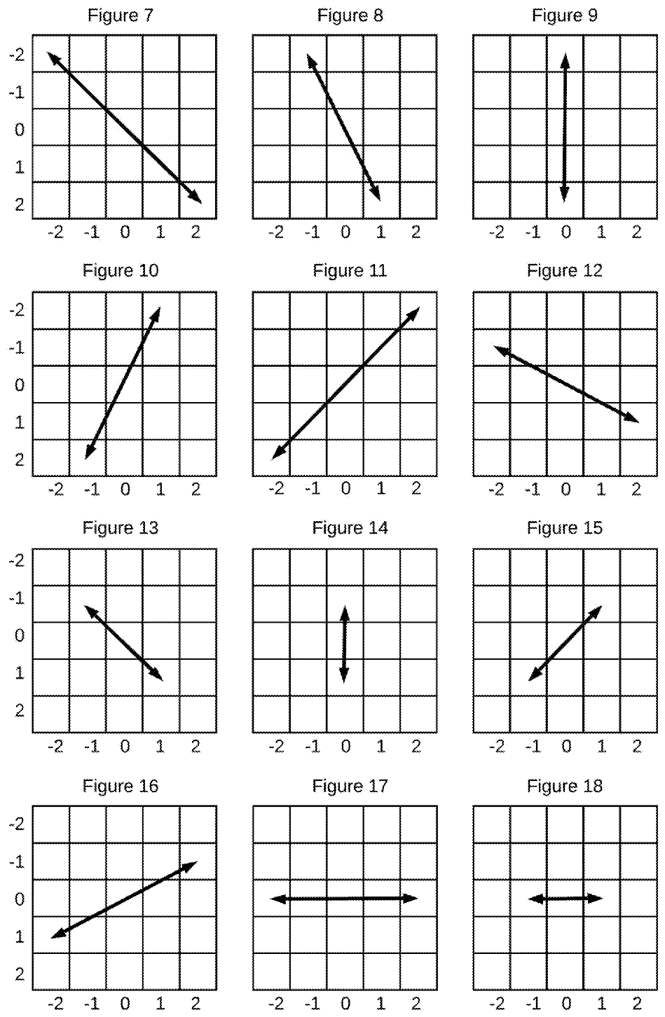

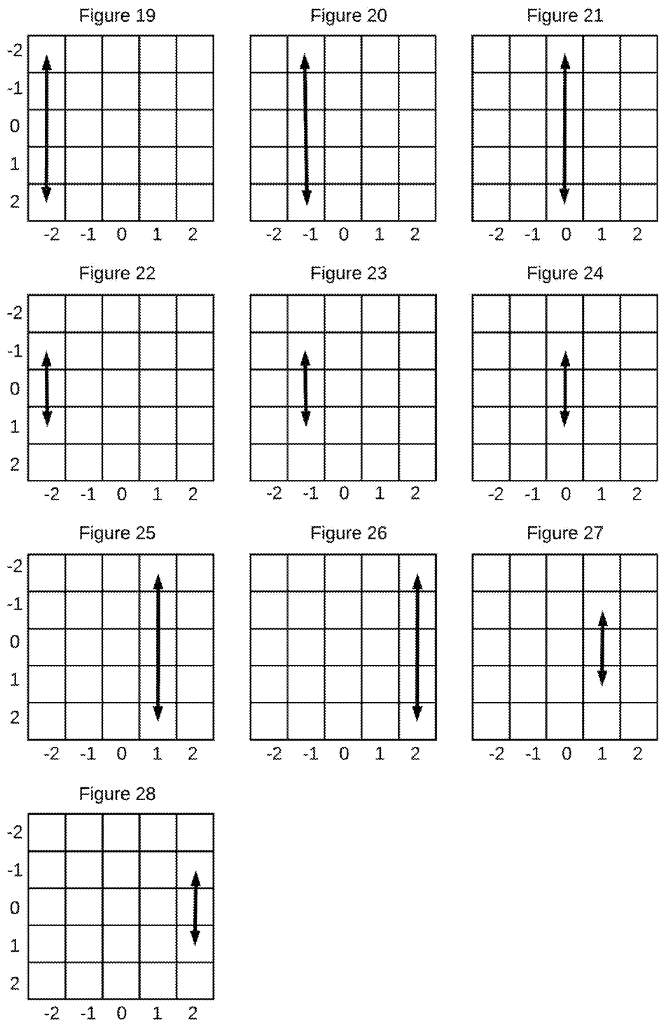

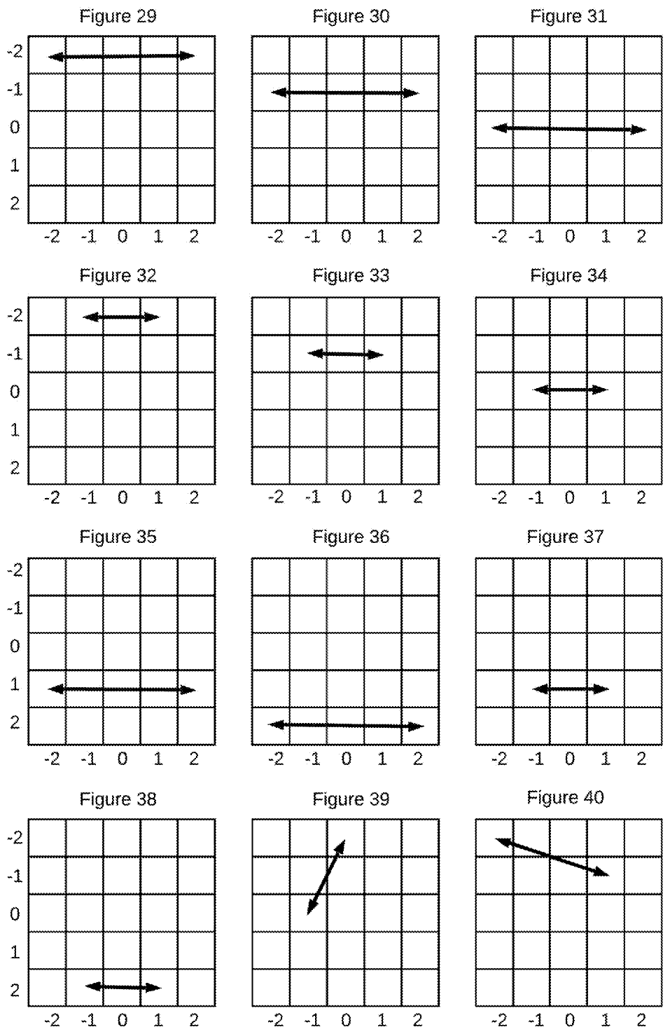

This can be achieved by selecting antonyms pairs from opposite sides of the 5×5 matrix. The opposite sides of the 5×5 matrix being those in the cells in which the arrow heads lie in FIGS. 7, 8, 9, 10, 11, 12, 16, 17, 19, 20, 21, 25, 26, 29, 30, 31, 35 and 36. This effectively is achieved by reflecting around the x-axis (words with the same level of dominance but opposite levels of affiliation), y-axis (words with the same level of affiliation but opposite levels of dominance) or both the x- and y-axis (words with opposite levels of affiliation and opposite levels of dominance). Additionally words can be selected from location which are not at opposite sides of the 5×5 matrix, as depicted in FIGS. 13 to 15, 18, 22 to 24, 27, 28, 32 to 34, and 37 to 40. FIGS. 39 and 40 for example show images of two alternate cells that antonyms could be selected from. Selection of antonyms from cells other than the paths identified in FIGS. 1 to 38 is not preferred, due to the antonyms not necessarily having an exactly opposite interpretation from a personality or behavioural perspective. The use of antonyms selected from non-preferred paths is considered to lead to less specific or sensitive psychological tests. It will be noted however, that all words selected, as indicated by the arrow heads, are in locations that are separated by 1 or more than one index units on the x and/or y axes, as is the case with FIGS. 1 to 38, while in FIG. 39 on the X-axis the location of arrow heads are in adjacent index units but on the Y-axis they are one unit index apart. This is contrasted with FIG. 40 where the location of selection is 2 index units apart on the X-axis, and only in adjacent Index units on the Y-Axis.

The benefits of this approach are:
a) Single words are shorter and easier to interpret. The time spent by the respondents analysing each question is reduced, thereby increasing compliance in completing the test.
b) The absence of a Likert-type scale reduces the respondent's time take consider their response. Having single words that are either applicable or not-applicable takes considerably less time to answer, thus improving compliance in completing the test.
c) The use of single words descriptors can be easily analysed using the inventive methodology and allow the responses to be graphed and analysed in two dimensions.

### Part 8: Method Applied

STEP 1: All antonymic pairs in the corpus are identified.
a. Observed that many tend to fall in three ways, which are called Diagonal (see FIGS. 7, 8, 10, 11, 12, 13, 15, 16, 39, 40), East-West (see FIGS. 17,18, 29 to 38) and North-South (see FIGS. 9, 14, 19 to 28). (Many also do not fall on these lines). There is no real directionality in the pairs—they are just from different sides of the 5×5 matrix.
b. Identify the subset of antonymic pairs that fall along these lines and do not use the antonymic pairs that don't fall on these lines.
c. Create additional antonyms by using words that can be made antonyms by adding a prefix or suffix. E.g
   i. gorm and gormless.
   ii. Friendly and unfriendly
   iii. Affiliative and disaffiliative
d. For each cell in the matrix, excluding the central cell or (0, 0), pick pairs so that all cells have 4 questions with a word in that cell. This will generate 96 questions in all.
e. Pairs may be generated
   i. Randomly
   ii. X diagonal, Y East west, Z north south, where X, Y and Z add up to 4.
f. If antonyms are exhausted (e.g. all used in previous steps), then pick any two words randomly, one from each cell.

STEP 2: Using a web page
a. Generate display of the antonymic pairs and ask the user to select the best option.
b. Importantly, must also ask whether if NEITHER word applies. See FIG. 42.

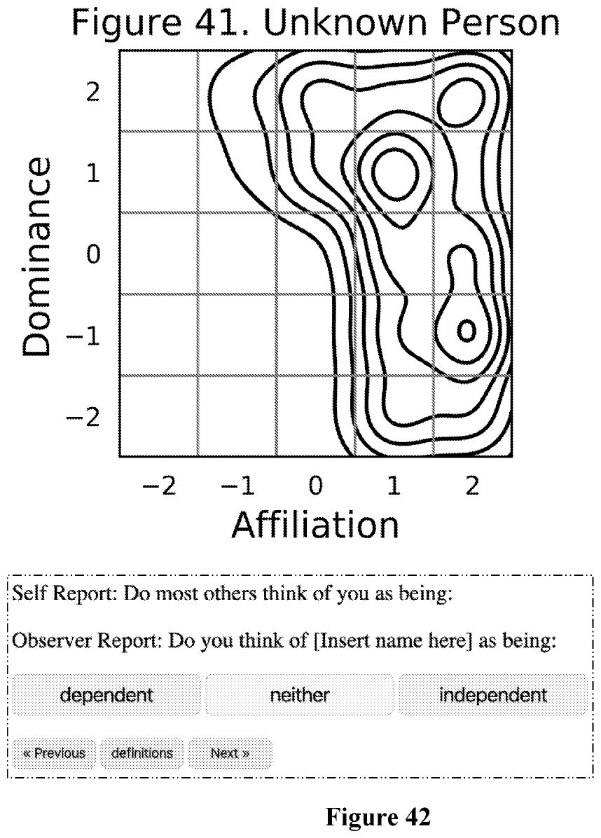

STEP 3: Generate a kernel density plot for the words selected. See FIG. 41.

STEP 4: Apply steps 1 to 3 above in applications
a. Psychiatric/psychological/neurobehavioral clinical diagnosis
b. Personality testing
c. Employment screening
d. Cultural surveys of organisations
e. 360 degree surveys of employees
f. Decision support

The above described embodiments and topologies can be useful in studies for use in commercial, clinical and scientific spheres. Of particular importance is the ability of the proposed topology to synthesise existing knowledge from taxonomies defined in terms of both adjectives (traits) and verbs (behaviours) within a single construct, and to facilitate the characterisation of personality, psychological, social, neurobiological, and linguistic concepts used in both current and future research.

Part 8: Results

The Part 8 Method will yield results which in FIG. 41 shows how they would be typically represented.

PART 9: A Modified PART 1 Catalogue Preparation of Relevant Words

Part 9: Method Applied

The author scanned the Word Net compilations of verbs, adjectives and nouns. Words and idioms were included in the catalogue if they related to any form of human interaction, including:
a. Words explicitly denoting dyadic, group, or societal interpersonal interaction.
b. Words typically thought of as being intrapersonal, but that may impact others at a future time (e.g. learn, research and invent).
c. Words that denote behaviour or personality, or emotions related to behaviour or personality.
d. Words that denote power.

In the course of cataloguing these words, it was observed that there is a subset of words that are related to behaviour and personality, but are not behaviour or personality per se. These words were categorised as words relating to ‘power’, or capacity to influence outcomes for another person. It was necessary to differentiate these words into a unique category in order to accurately categorise words and create a complete catalogue. Therefore, this formed the final category of words included in the catalogue.

Online thesauri (as in Part 1 above) were used to identify synonyms and antonyms for all words identified above, and scanned to identify any relevant additional words and idioms. The Oxford English Dictionary was used to classify the part of speech for each word.

The words were classified as being descriptive of behaviour, personality, power or emotion. Verbs describing observable actions were classified as behaviours. Adjectives describing patterns of behaviour were classified as personality traits. Abstract nouns describing mental states (feelings, moods, affect etc.) were classified as emotions. Nouns describing the capacity of one individual to influence the outcomes for another were classified as power.

Part 9: Results

20,688 words were identified as being descriptive of behaviour, emotion, personality and power. A summary of the words is shown in the Table 4 below:

TABLE 4
Summary of words denoting behaviour, emotion, personality and power.

| Part of | domain | | | | |
|---|---|---|---|---|---|
| Speech | behaviour | emotion | personality | power | Grand Total |
| adjective | | | 4,342 | 175 | 4,517 |
| idiom | 3,659 | 485 | 2,104 | 436 | 6,684 |
| noun | 1,442 | 2,601 | 1,041 | 1,337 | 6,421 |
| verb | 3,065 | 1 | | | 3,066 |
| Grand Total | 8,166 | 3,087 | 7,487 | 1,948 | 20,688 |

Reconciliations of the new catalogue were performed with prior art catalogues where available. The reconciliations showed that prior art catalogues included archaic words that are uncommon in modern dictionaries, such as ‘indeliberate’, ‘granousier’, ‘eremitic’ and ‘scientistic’. The reconciliation also showed words that were previously omitted, such as ‘adaptable’, ‘charismatic’, ‘perfectionist’ and, ‘withdrawn’. The existence of modern online word catalogues, online dictionaries and thesauri greatly assisted in the compilation of the proposed catalogue, the most comprehensive list of English-language words in personality research to date, numbering 20,688 words. With regard to the inventive procedures adopted, as well as the absolute number of words identified, the list can be considered to be unbiased and sufficient for the purposes of identifying a comprehensive taxonomy.

The cataloging process of Part 9 identified 1948 words descriptive of power, such as ‘rich’, ‘poor’, ‘skilled’, ‘unskilled’, ‘employed’, ‘unemployed’, ‘king’ and ‘servant’. Power has been demonstrated to moderate emotions, behaviour and personality.

PART 10: Confirm That Catalogue of PART 9 is Classified According to the Grid Taxonomy.

Part 10: Method

The inventor conducted a number of qualitative assessments in conjunction with a cognitive and behavioural neurologist, two clinical psychologists, one neuropsychologist, and individuals with a breadth of corporate, sporting, or legal expertise to prepare an initial allocation of catalogued words to cells in the grid taxonomy. This process was informed by the word placement allocations set by prior researchers of the Interpersonal Circumplex. Using the initial allocation, the inventor selected 35 words denoting behaviours, personality traits and emotions for each cell in the grid, approximately 2,600 words in total. A modified Delphi process using a panel of three clinical psychologists was conducted to allocate these words to one of the 25 cells in the grid taxonomy.

Subsequent to the Delphi process, the author allocated an additional 1,600 words and made a number of revisions to ensure that synonyms, conjugates and inflections were proximately located where appropriate. All revisions were confirmed by the neurologist and at least one psychologist. The resultant set of 4,200 words were then reviewed as a complete set by the neurologist and three psychologists until a consensus was achieved.

Part 10: Results

Consensus between the psychologists was achieved for the majority of words, confirming that the catalogued words can be successfully classified using the grid taxonomy. Table 5 below shows an example of an emotion, behaviour and personality trait for each cell in the grid taxonomy:

TABLE 5
Example emotions, behaviours and personality traits applicable to each cell in the grid taxonomy.
A more complete version of this is available at https://github.com/anthonymobbs/ptpeb.

| | | Affiliation | | | | |
|---|---|---|---|---|---|---|
| Dominance | Domain | −2 | −1 | 0 | 1 | 2 |
| 2 | Emotion | rage | conceit | determination | strength | triumph |
| | Behaviour | kill | bully | master | lead | inspire |
| | Personality | vicious | tough | superior | persuasive | charismatic |
| 1 | Emotion | hate | suspicion | concern | enjoyment | generosity |
| | Behaviour | reject | fight | make | give | nurture |
| | Personality | nasty | difficult | keen | agile | happy |
| 0 | Emotion | fear | discomfort | peace | warmth | affection |
| | Behaviour | vanish | delay | rest | welcome | collaborate |
| | Personality | peculiar | cool | plain | kind | reliable |
| −1 | Emotion | anguish | worry | lethargy | acceptance | belonging |
| | Behaviour | trembling | mope | snooze | conform | follow |
| | Personality | sad | anxious | dull | humble | selfless |
| −2 | Emotion | melancholy | weakness | boredom | duty | servility |
| | Behaviour | self-harm | collapse | slumber | capitulate | kowtow |
| | Personality | morose | cowardly | passive | obedient | obsequious |

The thesauri did not frequently identify synonymic associations between the words descriptive of emotions, behaviours and personality; for example, the words kill (behaviour) and murderer (personality) were not synonymously related. The reference thesauri did however nominate ‘killer’ (personality) and ‘murderer’ (personality) as synonyms. ‘Kill’ and ‘killer’ can be linked by virtue of having the same linguistic stem. The linking of stem words was performed manually in this paper, however it could be automated in future. By supplementing the thesauri derived synonyms with manually linked stem words, a robust association between emotions, behaviours and personality traits was achieved.

Some emotions are known to give rise to physiological changes, such as happiness, love, pride, anger, fear, anxiety, shame, sadness, depression, disgust, contempt, and envy. It was noted that these emotions are located on the outer edge of the grid taxonomy. Strong emotions are thought to prompt the body to undertake urgent and impactful behaviour, consistent with the colocated behaviours at the extremity of the grid taxonomy.

Historically, it has been acknowledged that relationships exist between personality, emotions and behaviour, although the exact nature of these relationships remains unclear. The newly described grid taxonomy provides a common framework by which to understand emotions, behaviour and personality, and facilitate future investigation of the causal associations between them.

### PART 11: Automate Encoding of Words in Catalogue Using a Spring-Based Network Analysis.

Part 11: Method

A Python computer program (see Appendix 2) was developed to implement the following steps:
1. The synonyms and antonyms for all catalogued words were obtained from the Oxford and Merriam-Webster thesauri.
2. First iteration
   a. For uncoded words in the catalogue with 100% (threshold) of their synonyms previously coded, the equilibrium position was calculated such that the forces of attraction between synonymic words and repulsion between antonymic words were minimised. Hooke's Law was used to calculate the forces of attraction and repulsion. Hooke's Law states that the force needed to extend or compress a spring is proportional to the extension or compression from the resting position. Synonyms that are distant from each other will experience a strong force of attraction whereas synonyms that are close together will experience no force. Conversely, antonyms that are close together will experience a strong repulsive force and antonyms that are distant from each other will experience no repulsive force.
   b. Step a. was repeated by successively reducing the threshold (initially set at 100%) by 1% until all words in the catalogue were encoded.
3. Subsequent iterations
   a. The equilibrium position for each word in the catalogue was calculated in alphabetical order allowing words to reposition within the grid.
   b. This process was repeated until variations of the encoded words ceased.

Part 11: Results

A total of 540,592 synonymic pairs and 96,890 antonymic pairs were identified between words in the catalogue. Eight iterations of Step 3 of the method were required until equilibrium was achieved. Table 6 below shows the number of words within each cell of the grid taxonomy:

TABLE 6
Frequency of words in each cell of Grid Taxonomy

| COUNT | | | | | | | |
|---|---|---|---|---|---|---|---|
| of word | domi- | affiliation | | | | | Grand |
| domain | nance | −2 | −1 | 0 | 1 | 2 | Total |
| behaviour | 2 | 413 | 286 | 147 | 191 | 99 | 1,136 |
| | 1 | 539 | 1,749 | 2,056 | 1,076 | 56 | 5,476 |
| | 0 | 94 | 604 | 167 | 184 | 148 | 1,197 |
| | −1 | 7 | 35 | 31 | 61 | 65 | 199 |
| | −2 | 9 | 8 | 4 | 32 | 67 | 120 |
| emotion | 2 | 114 | 132 | 81 | 22 | 98 | 447 |
| | 1 | 325 | 351 | 407 | 194 | 49 | 1,326 |
| | 0 | 217 | 278 | 73 | 117 | 120 | 805 |
| | −1 | 47 | 101 | 24 | 32 | 57 | 261 |
| | −2 | 64 | 50 | 39 | 35 | 47 | 235 |
| personality | 2 | 199 | 361 | 642 | 38 | 132 | 1,372 |
| | 1 | 795 | 728 | 615 | 288 | 104 | 2,530 |
| | 0 | 476 | 1,373 | 357 | 262 | 119 | 2,587 |
| | −1 | 150 | 226 | 97 | 58 | 79 | 610 |
| | −2 | 20 | 102 | 66 | 86 | 74 | 348 |
| Grand Total | | 3,469 | 6,384 | 4,806 | 2,676 | 1,314 | 18,649 |

A qualitative review by the method of Part 11, of the word placement by the spring-based network method confirmed that the method accurately placed the majority of words.

The spring-based network approach of Part 11 was able to encode all words in the catalogue not previously encoded in Part 10. A review of the encoded words suggested that the process was accurate and satisfactory for the purpose of analysing existing psychological and social constructs.

### PART 12: A Further Improved Psychometric Test:

Part 12: Method

To be comprehensive, it was determined that the psychological test must ask questions that span the entire grid taxonomy. The grid taxonomy has an odd number of cells, 25. Dipole questions can only cover an even number of cells. The centre cells of the taxonomy (0, 0) is theorised to be the most predictable within the taxonomy, as it covers intrapersonal and adominant personality descriptors such as ‘ordinary’, ‘average’ and ‘common’ that may encompass larger populations by definition. After excluding the centre cell, 24 cells remained, requiring a minimum of 12 dipole questions to span the whole taxonomy. From this it was inferred that the minimum number of questions required for a comprehensive psychological test is 12.

To determine the optimal configuration of the 12 questions required for a comprehensive psychological test, it is noted that sensitivity and specificity are widely used statistical measures for the performance of binary classification tests. The concept of sensitivity measures the proportion of correctly identified positives, and specificity measures the proportion of correctly identified negatives. To achieve high levels of sensitivity and specificity, a parsimonious test must ask questions that maximally distinguish the concepts under consideration. For example antonymic binary choice questions, such as ‘are you usually friendly or unfriendly?’ are preferable when compared with near synonymic binary choice questions, such as ‘are you usually friendly or polite?’.

In a grid of 24 cells, there are 24P24≈10²³ permutations of binary questions that could be asked of the respondent. It is not possible with modern computational techniques to test all 10²³ permutations in order to identify the combinations that maximise the overall contrast, therefore, a simulation and alpha-beta pruning approach was used to determine which combinations maximise the average distance between the possible antonymic binary pairs.

Part 12: Results

In total, 5 billion simulations were run, revealing that the average distance between antonymic pairs was maximised when antonymic pairs were selected from opposite sides of the grid and reflected through the origin (FIG. 4). For example, the selection of antonymic word pairs such as blissful (2, 2) and despondent (−2, −2) have maximal contrast, and are located on opposite sides of the grid (FIG. 4.e). The catalogue contained approximately 3,400 antonymic adjectival word pairs that were maximally contrasting. The alpha-beta pruning refinement revealed that 16 of the 10²³ permutations maximised the average distance between the possible antonymic binary pairs (see FIG. 4). Of these 16 combinations, only one had sufficient words catalogued to facilitate a psychological test (FIG. 4). Therefore, this combination was selected as the basis of the new psychological test. For example, the selection of antonymic word pairs such as blissful (2, 2) and despondent (−2, −2) maximise the contrast, given that these words are located on opposite sides of the grid. The simulation confirmed that the center cell (0, 0) was not selected for tests that maximised the average distance between the possible antonymic binary pairs. Further, there were few antonymic pairs with an endpoint at (0, 0).

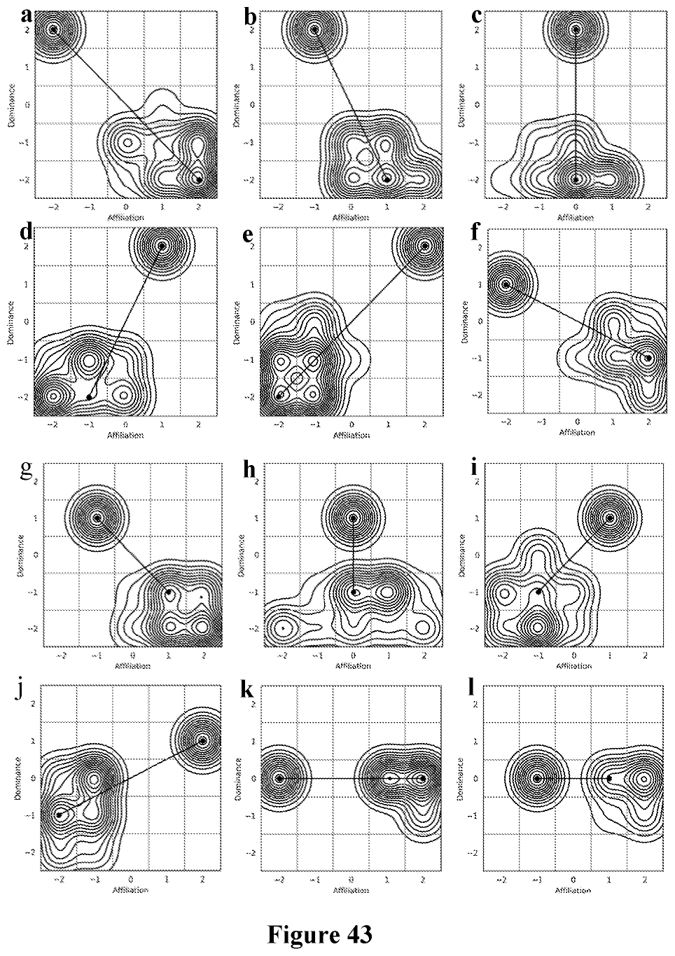

For tabulation of these results reference is made to FIG. 43 which shows the binary pairs maximising overall contrast of the psychological test. For each of the 12 graphs, the blue and orange kernel density plots represents each side of the antonymic dipole. The kernel density plots are representative of the 299 observations (99.999999th percentile) out of 4 billion simulations that average distance between the 12 pairs, maximising the overall contrast of the psychological test. The lines show on each graph represent the binary pairs that have a sufficient number of antonyms identified in the reference thesauri to allow the construction of a psychological test. The diamond at location (0, 0) represents the point of reflection about which the antonymic pairs are reflected.

A comparison of the psychological test of Part 12, with prior art tests is shown in table 6 below:

TABLE 6
Facets of Psychological Test Efficacy
for several psychological tests and constructs

| | Average | | | | |
|---|---|---|---|---|---|
| | Response | Average | | | |
| | Concen- | Question | Complete- | | |
| Construct | tration | Clarity | ness | Contrast | Dipoles |
| ADHD | 0.27 | 0.57 | 0.04 | 0.00 | 0 |
| ICD-11 | 1.00 | 1.00 | 0.20 | 0.00 | 0 |
| DSM-5 | 1.00 | 1.00 | 0.48 | 0.00 | 0 |
| Dark Triad | 0.22 | 0.45 | 0.12 | 1.00 | 1 |
| Need for Cognition | 0.22 | 0.49 | 0.08 | 4.00 | 1 |
| Physical | 0.24 | 0.42 | 0.08 | 4.24 | 1 |
| Attractiveness | | | | | |
| Cognitive Failure | 0.32 | 0.66 | 0.08 | 1.41 | 1 |
| Optimism | 0.37 | 0.54 | 0.04 | 3.60 | 1 |
| IPIP NEO | 0.26 | 0.46 | 0.36 | 2.50 | 5 |
| HEXACO | 0.26 | 0.46 | 0.28 | 2.26 | 6 |
| PART 12 METHOD | 1.00 | 1.00 | 0.96 | 3.90 | 12 |

For a psychological taxonomy consisting of a square grid of 25 cells, the minimum number of questions required for a comprehensive psychological test is 12. Whereas, a psychological test consisting of antonymic dipoles taken from opposite sides of the grid taxonomy, when reflected about the origin, maximises the available contrast. Whilst such a test would be highly efficient, it is unlikely to achieve the statistical power required for discriminatory testing. To achieve the requisite level of statistical power, it is likely that multiple iterations of the 12 questions will be required. At this point in time the number of iterations required to achieve the level of statistical power suitable for particular applications is not yet known, however, it is expected by simply conducting a number of iterations the user will be able to identify when equilibrium is achieved.

From the taxonomies which result from Parts 9 to 12 above, the following OBSERVER Report Questionnaire and SELF Report Questionnaire can be precipitated. The OBSERVER Report is comprised of adjectival descriptors of personality that are observed by third parties, whereas the SELF Report consists of nouns descriptive of emotions that are felt by a subject when doing a respective test. Such Questionnaires can be given by computer or mobile device means, or as a paper version.

Observer Report:

| | | | | | |
|---|---|---|---|---|---|
| despondent | □ | neither | □ | hopeful | □ |
| negative | □ | neither | □ | positive | □ |
| pessimist | □ | neither | □ | optimist | □ |
| wretched | □ | neither | □ | excellent | □ |
| cheerless | □ | neither | □ | cheerful | □ |
| downbeat | □ | neither | □ | upbeat | □ |
| sad | □ | neither | □ | happy | □ |
| joyless | □ | neither | □ | joyful | □ |
| disconnected | □ | neither | □ | connected | □ |
| uncooperative | □ | neither | □ | cooperative | □ |
| unsociable | □ | neither | □ | sociable | □ |
| untrusting | □ | neither | □ | trusting | □ |
| disagreeable | □ | neither | □ | agreeable | □ |
| intolerant | □ | neither | □ | tolerant | □ |
| ungrateful | □ | neither | □ | grateful | □ |
| unreliable | □ | neither | □ | reliable | □ |
| insensitive | □ | neither | □ | sensitive | □ |
| tough | □ | neither | □ | tender | □ |
| uncaring | □ | neither | □ | caring | □ |
| unkind | □ | neither | □ | kind | □ |
| afraid | □ | neither | □ | unafraid | □ |
| cowardly | □ | neither | □ | courageous | □ |
| indirect | □ | neither | □ | direct | □ |
| unadventurous | □ | neither | □ | adventurous | □ |
| agitated | □ | neither | □ | calm | □ |
| discontent | □ | neither | □ | content | □ |
| doubtful | □ | neither | □ | confident | □ |
| indecisive | □ | neither | □ | decisive | □ |
| inattentive | □ | neither | □ | attentive | □ |
| unrealistic | □ | neither | □ | realistic | □ |
| untalkative | □ | neither | □ | talkative | □ |
| untidy | □ | neither | □ | tidy | □ |
| aggressive | □ | neither | □ | peaceful | □ |
| dishonest | □ | neither | □ | honest | □ |
| inflexible | □ | neither | □ | flexible | □ |
| unhelpful | □ | neither | □ | helpful | □ |
| arrogant | □ | neither | □ | humble | □ |
| demanding | □ | neither | □ | undemanding | □ |
| judgemental | □ | neither | □ | non-judgemental | □ |
| selfish | □ | neither | □ | unselfish | □ |
| unambitious | □ | neither | □ | ambitious | □ |
| unassertive | □ | neither | □ | assertive | □ |
| unenergetic | □ | neither | □ | energetic | □ |
| unmotivated | □ | neither | □ | motivated | □ |
| disinterested | □ | neither | □ | interested | □ |
| inactive | □ | neither | □ | active | □ |
| unobservant | □ | neither | □ | observant | □ |
| unproductive | □ | neither | □ | productive | □ |

Self Report:

| | | | | | |
|---|---|---|---|---|---|
| despair | □ | neither | □ | hope | □ |
| despondency | □ | neither | □ | jubilation | □ |
| gloom | □ | neither | □ | exuberance | □ |
| pessimism | □ | neither | □ | optimism | □ |
| cheerlessness | □ | neither | □ | cheerfulness | □ |
| misery | □ | neither | □ | bliss | □ |
| sorrow | □ | neither | □ | joy | □ |
| unhappiness | □ | neither | □ | happiness | □ |
| disconnection | □ | neither | □ | connection | □ |
| disharmony | □ | neither | □ | harmony | □ |
| disorder | □ | neither | □ | order | □ |
| unfairness | □ | neither | □ | fairness | □ |
| disagreeableness | □ | neither | □ | agreeableness | □ |
| disloyalty | □ | neither | □ | loyalty | □ |
| intolerance | □ | neither | □ | tolerance | □ |
| ungratefulness | □ | neither | □ | gratefulness | □ |
| callousness | □ | neither | □ | tenderness | □ |
| cruelty | □ | neither | □ | compassion | □ |
| hate | □ | neither | □ | love | □ |
| unkindness | □ | neither | □ | kindness | □ |
| cowardice | □ | neither | □ | bravery | □ |
| fearfulness | □ | neither | □ | fearlessness | □ |
| timidity | □ | neither | □ | boldness | □ |
| weakness | □ | neither | □ | strength | □ |
| discomfort | □ | neither | □ | comfort | □ |
| dissatisfaction | □ | neither | □ | satisfaction | □ |
| uncertainty | □ | neither | □ | assurance | □ |
| unease | □ | neither | □ | ease | □ |
| inequality | □ | neither | □ | equality | □ |
| instability | □ | neither | □ | stability | □ |
| unfamiliarity | □ | neither | □ | familiarity | □ |
| unreasonableness | □ | neither | □ | reasonableness | □ |
| disapproval | □ | neither | □ | approval | □ |
| displeasure | □ | neither | □ | pleasure | □ |
| impatience | □ | neither | □ | patience | □ |
| inflexibility | □ | neither | □ | flexibility | □ |
| disobedience | □ | neither | □ | obedience | □ |
| conceit | □ | neither | □ | modesty | □ |
| pride | □ | neither | □ | humility | □ |
| selfishness | □ | neither | □ | unselfishness | □ |
| apathy | □ | neither | □ | enthusiasm | □ |
| inertia | □ | neither | □ | drive | □ |
| lethargy | □ | neither | □ | energy | □ |
| vulnerability | □ | neither | □ | invulnerability | □ |
| disinterest | □ | neither | □ | interest | □ |
| incompetence | □ | neither | □ | competence | □ |
| purposelessness | □ | neither | □ | purposefulness | □ |
| unproductiveness | □ | neither | □ | productiveness | □ |

The above described topologies are useful in:
1. Bayesian personality test
   a. Where the test may be presented in two ways
      i. Self-report
      ii. Observer report
   b. A fixed number of initial questions can be asked (approximately 24)
      i. These questions will be drawn from the catalogue described above of personality descriptive verbs, adjectives, nouns idioms and emotions.
      ii. The method of selection of the initial question would most likely follow Part 8.
      iii. The chance of any two individuals being asked the same set of initial questions will be infinitesimally small (therefore deterring individuals being surveyed from copying the survey results of another participant or attempting to learn all questions in advance, i.e. reducing false-positive rate).
   c. The initial response will form the basis of subsequent questions, which are, again, variable.
      i. Subsequent questions will be derived from a Bayesian engine that can determine the next most appropriate question to ask.
         1. The Bayesian engine can identify candidate psychological constructs, which are then tested by subsequent questions.
         2. The Bayesian engine can rank order the candidate psychological constructs in order of statistical significance.
         3. The Bayesian engine can identify combinations of psychological constructs that are unusual in combination—as a means of identifying candidates who are falsely scoring their abilities.
         4. The Bayesian engine can identify combinations of psychological constructs that are ‘too good’ in combination—as a means of identifying candidates who are falsely scoring their abilities. E.g. the candidate is ‘perfect’ in all areas and without any negative traits.
      ii. The chance of any two individuals being asked the same set of subsequent questions will be infinitesimally small (therefore can't be learned in advance)
      iii. The system can identify relevant clusters of psychological traits
      iv. The system can stop when a statistically significant determination of the individuals most likely personality attributes is established.
      v. If the individual has multiple psychological constructs, the system may rank them by order of importance.
      vi. Depending upon the degree of statistical significance required, the system can ask more or less questions to satisfy the application. For example: In an employment context, a very high level of statistically significant findings would be indicated. This is compared to a role without people management responsibilities and/or little customer interaction required, where a much lower level of statistically significant results would typically be required.
      vii. The test can identify deficits that the candidate has relative to the role they are seeking to be employed in.
      viii. The system can retain all previous collected results. These can be input to a Bayesian engine that can further refine the approach to conducting surveys in the future.
      ix. Such a system can have cultural, societal and inter-user sensitivity that coincides to the dynamic alteration of language itself, i.e. the Bayesian approach utilised can be considered a form of artificial intelligence.
      x. The system can carry an intrinsic capability to define personality types in whole, based upon collective results, clusters and patterning, according to a particular cultural, societal or interpersonal context. BY this it is meant that the system can have an ongoing learning capability of the system.
   d. The results of the personality test can be presented on a two dimensional grid, typically a five by five grid, but smaller or larger grids can be used such as three by three or seven by seven, three by five, three by seven, five by seven.
   e. The results may also identify the relevance of the results using a range of historically significant personality constructs, for example, Five Factor Model, Diagnostic and Statistical Manual Personality Disorders, Hare Psychopathy, Emotional Intelligence, Empathy, Ryff's Wellness Scale and several others.

The advantages for these two test regimes are that while existing tests focus on personality descriptive adjectives, the claimed invention embraces verbs, adjectives, nouns, idioms and emotion. As such the specific tests offer a Bayesian version which has the following characteristics:
i. Impossible for candidates to learn in advance
ii. Inbuilt detection of inconsistent responses
iii. Improved psychometric testing of for employment suitability testing
iv. Reduced number of questions need be asked to establish a meaningful result
v. Perpetual analysis and system learning capability, and, therefore, adaptability.

The above described topology allows for the derivation of personality from voice or text, by the ingestion or processing of text typically sourced from published or communicative materials, such as: Emails; Text or SMS messages; Social media; Blogs; Speeches as described in Part 7 above, or otherwise; Books; Articles; Newspapers; Chat bots; Text transformed from voice recognition systems; and Transcripts.

Such text can be analysed using lexical approach, whereby words within the text are categorised according to the catalogue. Words within each relevant cell of the five by five matrix are accumulated and the results of the personality test presented on a two dimensional grid, preferably a five by five grid, but may be three by three or seven by seven, three by five, three by seven, five by seven.

In respect of the psychometric tests of Part 5, 8 or 12 described above, these can be given by means of one or more than one of: an app, an application, a phone, a mobile device, a web based application, a website, a paper based questionnaire.

Where ever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

It will be understood that the invention disclosed and defined herein extends to all alternative combinations of two or more of the individual features mentioned or evident from the text. All of these different combinations constitute various alternative aspects of the invention.

While particular embodiments of this invention have been described, it will be evident to those skilled in the art that the present invention may be embodied in other specific forms without departing from the essential characteristics thereof. The present embodiments and examples are therefore to be considered in all respects as illustrative and not restrictive, and all modifications which would be obvious to those skilled in the art are therefore intended to be embraced therein.

1. A method of categorising words and/or text wherein the following steps are performed:
a) compiling a catalogue of selected words of a language which are identified and selected from at least one dictionary and which are descriptive of intrapersonal behaviours and interpersonal interactions, and said selected words being of one of, or combinations of two or more of, or all of, the following types: verbs, adjectives, nouns and idioms;
b) identifying synonyms for each one of said selected words from at least one thesaurus;
c) identifying archetypal words from the respective groups of one selected word and its respective synonyms;
d) rating said archetypal words with scores relating to affiliation and dominance thereby producing a matrix;
e) applying ratings to all of said selected words and said synonyms.

2. A method as claimed in claim 1, wherein said matrix is one of: three by three or a five by five or seven by seven, or three by five, or three by seven, or five by seven.

3. A method as claimed in claim 2, wherein said matrix when it includes an axis of three, has index values of −1, 0, +1; when it has and axis of five, has index values of −2, −1, 0, +1, +2; or when it has an axis of seven, has index values of −3, −2, −1, 0, +1, +2, +3.

4. A method as claimed in claim 1, wherein said matrix is a five by five matrix, and has indexes of −2, −1, 0, +1, +2.

5. A method as claimed in claim 1, wherein said method is modified by synonyms being replaced by antonyms.

6. A method of categorising words and/or text wherein the following steps are performed:
a) compiling a catalogue of selected words of a language which are identified and selected from at least one dictionary and which are descriptive of intrapersonal behaviours and/or interpersonal interactions, and the selected words being of one of, or combinations of two or more of, or all of, the following types: verbs, adjectives, nouns and idioms;
b) identifying antonyms for each one of the selected words from at least one thesaurus;
c) identifying archetypal words from the respective groups of one selected word and its respective antonyms;
d) rating the archetypal words with scores relating to affiliation and dominance thereby producing a matrix;
e) applying ratings to all of the selected words and the antonyms.

7. A method as claimed in claim 6, wherein said matrix is one of: three by three or a five by five or seven by seven, or three by five, or three by seven, or five by seven.

8. A method as claimed in claim 7, wherein said matrix when it includes an axis of three, has index values of −1, 0, +1; when it has and axis of five, has index values of −2, −1, 0, +1, +2; or when it has an axis of seven, has index values of −3, −2, −1, 0, +1, +2, +3.

9. A method as claimed in claim 6, wherein said matrix is a five by five matrix, and has indexes of −2, −1, 0, +1, +2.

10. A method as claimed in claim 6, wherein said antonyms are in a 5×5 matrix.

11. A method as claimed in claim 6, wherein said antonyms are selected from said matrix by being separated by at least one index unit on at least one of the X-axis and or Y-axis.

12. A method as claimed in claim 6, wherein said antonyms are used in a test regarding personality and or behaviour and or emotion.

13. A method as claimed in claim 12, wherein a subject of said test is provided said antonyms and is asked for a reaction to them, through one or more than one of: an app, an application, a phone, a mobile device, a web based application, a website, or a paper based questionnaire.

14. A method as claimed in claim 12, wherein the subject is given a choice of “NEITHER” of the words to choose from.

15. (canceled)

16. A personality and or behaviour classification system comprising analysis of the words utilised or parsed by a subject, said system including testing said subject to collect parsed words or collecting the words (by voice to text or transcripts) and or writings of said subject, analysing said utilised or parsed words by means of the categorising method of claim 5, whereby said utilised or parsed words are said selected words and or said antonyms of said selected words.

17. A system as claimed in claim 16, wherein said words are provided by a subject through one or more than one of: an app, an application, a phone, a mobile device, a web based application, a website, a paper based questionnaire.

18. A system as claimed in claim 16 wherein the words are collected by voice to text or transcripts.

19. A system as claimed in claim 16, including reducing voice to text, or review of transcripts of said speech, and applying said method or matrix to key words used in said text and or transcript.

20. A system as claimed in claim 19, wherein said speech or words are in a language other than the language used in said method or matrix, words are translated into the language used in said method or matrix.

21. A system as claimed in claim 20, wherein said language, dictionary and or thesaurus is, or is applicable to, one of the following languages: English, French, German, Spanish, Portuguese, Chinese, Japanese, Korean, Indian, Arabic, Greek, or any other language translatable by Google Translate.

22. A method of analysing speech by means of the method or matrix of claim 1, said method including reducing voice to text, or review of transcripts of said speech, and applying said method or matrix to key words used in said text and or transcript.

23. A method as claimed in claim 1, wherein when said speech is in a language other than the language used in said method or matrix, said text or said transcript is translated into the language used in said method or matrix.

24. A method or matrix as claimed in claim 1, wherein said language, dictionary and or thesaurus is, or is applicable to, one of the following languages: English, French, German, Spanish, Portuguese, Chinese, Japanese, Korean, Indian, Arabic, Greek, or any other language translatable by Google Translate.

25.-28. (canceled)
